(19) United States
(12) Patent Application Publication
(10) Pub. No.: US 2010/0233737 A1
(43) Pub. Date: Sep. 16, 2010

(54) MARKER SPECIFIC TO AN OXIDATIVE DEGRADATION OF TISSUES CONTAINING TYPE III COLLAGEN, MEANS AND METHODS AND KITS FOR THE DIAGNOSIS, MONITORING OR PROGNOSIS OF PATHOLOGIES TARGETED BY THIS MARKER

(76) Inventors: Patrick Garnero, Gaujac (FR); Nadine Charni-Ben Tabassi, Villeurbanne (FR)

Correspondence Address: PEARNE & GORDON LLP, 1801 EAST 9TH STREET, SUITE 1200, CLEVELAND, OH, 44114-3108, US

(21) Appl. No.: 12/515,012

(22) PCT Filed: Nov. 12, 2007
(86) PCT No.: PCT/EP07/62196

§ 371 Date: Apr. 19, 2010

(30) Foreign Application Priority Data
Nov. 15, 2006 (FR) 06/09983

(51) Int. Cl. G01N 33/53; C07K 5/10; C07K 7/06; C07K 7/08; C07K 14/00; C07H 21/00; C07K 16/00

(52) U.S. Cl. 435/7.92; 530/330; 530/329; 530/328; 530/327; 530/326; 530/325; 530/324; 536/23.1; 530/387.1

(57) ABSTRACT

Novel peptide marker, specific to the oxidative degradation of tissues containing type III collagen, preferably to nitrosylation of the type III collagen. This marker is defined of at least one peptide sequence of 5 to 25 amino acids (AAs) including a sub-sequence QYDSYD in which at least one of the Ys is nitrosylated and Q corresponds not only to glutamine but also to pyroglutamic acid. This marker is simple, sensitive and reliable and expedites the clinical information for obtaining oxidative degradation of tissues containing type III collagen (O.D.T.Coll III) pathologies. This marker allows better monitoring, prognosis and treatment of the O.D.T.Coll III pathologies. This invention concerns also elements of detection of the marker, methods and kits which allow, on one hand, the early, reliable, efficient and economical diagnosis of the pathologies targeted and, on the other hand, improved monitoring and prognosis of the O.D.T.Coll III pathologies.

### TECHNICAL FIELD

The field of the invention is that of tools and methods for the diagnosis, monitoring and prognosis of diseases which are accompanied by an oxidative degradation of tissues containing type III collagen. These may be in particular osteoarticular diseases such as in particular arthrosis, rheumatoid polyarthritis, psoriasic arthritis, gout, chondrocalcinosis, Yersinia-induced arthritis, pyrophosphate arthritis, septic arthritis or disorders linked to the intervertebral discs such as spondylarthritis.

More precisely, the invention relates to the means and methods for evaluating the degradation of the synovial tissue, in particular synovial type III collagen, under the effect of an oxidative stress specific to these diseases, in particular the osteoarticular diseases.

More precisely still, the invention relates to, as products per se, peptide biological markers, the elements recognizing these markers (antibodies), the methods and kits for detecting said markers using said elements, for diagnosis-, monitoring- and prognosis-type uses for the abovementioned diseases, in particular the osteoarticular diseases.

The invention also relates to the means for obtaining peptide markers and antibodies recognizing said markers, these means being for example nucleic sequences.

### PRIOR ART—POSITION REGARDING THE PROBLEM

The diagnosis, treatment, monitoring and prognosis of diseases which are accompanied by an oxidative degradation of tissues containing type III collagen, is a major public health challenge.

As examples of these diseases, there can be mentioned cancer, neurodegenerative diseases such as Parkinson's disease or Alzeimer's disease etc.

Type III collagen is a homotrimer of identical alpha 1 (III) collagen chains, bordered by telopeptide linear regions. Type III collagen is located with type I collagen in the skin and the vascular tissues. Type III collagen is the major fibrillar collagen of the skin and of the vascular tissue. As regards the joint, type III collagen is the major collagen of the synovial membrane.

The inflammatory diseases such as the osteoarticular diseases, represent a significant proportion of this type of diseases which are accompanied by an oxidative degradation of tissues containing type III collagen.

Arthrosis and rheumatoid polyarthritis (arthritis) are two of the most common osteoarticular diseases.

Arthrosis can be due to age or trauma. Arthrosis causes severe lesions in the cartilage, immobility and bone excrescences of the joint (osteophytes). This disease affects the entire joint, including the cartilage, the synovial membrane and the bones connected to each other by this joint. The degradation of the cartilage in arthrosis is characterized by two phases: a biosynthesis phase during which the chondrocytes attempt to repair the damaged extracellular matrix and a degradation phase where the enzymes produced by the chondrocytes digest the matrix. The biosynthesis activity is then unequal to the catabolic activity, which leads to an inhibition of the synthesis of the matrix and to an acceleration of the erosion of the cartilage. Arthrosis is therefore an exaggeration of the bone remodelling process in the joint. In normal adult cartilage, the chondrocytes synthesize the constituents of the matrix slowly. The renewal of the matrix is strictly regulated: there is a balance between synthesis and degradation.

Rheumatoid polyarthritis is an auto-immune disease causing chronic inflammation of the joints and destruction of the cartilage, bones, tendons and ligaments. The inflammation originates from a failure of the immune system which does not recognize the organism's own cells and destroys them. In rheumatoid polyarthritis, the inflammation of the synovial membrane causes excessive production of synovial fluid and enzymes by the synoviocytes. The latter are at the origin of the degradation of the cartilage and the bone. When this situation becomes chronic, this results in inflammation, pain, and, possibly, a mobility problem.

It is difficult to diagnose these diseases rapidly. In fact, the pain and stiffness of the joint appear only late and radiography makes it possible to detect the disease only at an advanced stage of the latter. There is therefore a major benefit in finding markers (for example in the serum or urine) specific to the renewal of the constituents of the joint. The use of these specific biological markers is all the more beneficial as it constitutes a non-invasive technique which makes it possible to detect the disease early, monitor its evolution and establish a prognosis.

The biological or biochemical markers of joint renewal can be bone markers specific to bone formation or bone resorption, cartilaginous markers specific to the formation of the cartilage or degradation of the cartilage, or also markers of the synovial membrane specific to the synthesis or degradation of the latter.

Within the framework of the present invention, there is more particular interest in the biological or biochemical markers of the synovial membrane.

Moreover, it is known that the oxidation of the proteins by hyperoxidizing species is a phenomenon involved in the pathogenesis of numerous diseases such as the neurodegenerative diseases, cancers, the arteriosclerosis, cataracts, dysplasia, dystrophy and the inflammatory diseases including in particular the osteoarticular diseases as well as in ageing.

The hypetoxidizing species include nitrogen monoxide (NO). NO has a regulatory function but also, when it is overexpressed under inflammatory conditions, exerts a cytotoxic effect inducing oxidative stress. In its regulatory function, it acts as a protective agent which suppresses cell proliferation and protein synthesis. It thus makes it possible to lessen the inflammatory reaction. But NO can also act as an agent of oxidative stress by inhibiting the regeneration of the tissues and the synthesis of new matrix proteins.

The hyperoxidizing power of NO and of its by-products of reaction with the superoxide anion O₂⁻, is at the origin of its cytotoxicity.

In the case of osteoarticular diseases of the rheumatoid polyarthritis or arthrosis type, the overexpression of NO by the chondrocytes, the macrophages and the synovial tissue in terms of inflammation, is significant but is not specific to joint pathologies.

It is moreover known that the oxidative stress induced by NO and its peroxidizing by-products, in particular has the effect of nitrolysation (or nitration), of aromatic compounds such as the aromatic amino acids phenylalanine, tyrosine and tryptophan, which are present in numerous peptides and proteins, in particular in the joints. A well-known joint protein is type I, II, III, VI, IX or XI collagen.

It has therefore been envisaged to choose nitrotyrosine as a marker of oxidative stress associated with the diseases leading to damage due to oxidation.

Patent Application PCT WO-A-96/04311 thus describes anti-nitrotyrosine antibodies capable of recognizing and binding to nitrotyrosine and to nitrotyrosine residues within proteins. These anti-nitrotyrosine antibodies are described as useful in methods for detecting various pathological conditions and, in particular, inflammations and tumours. These anti-nitrotyrosine antibodies are also useful as a means of targeting therapeutic agents in tissue sites subject to damage resulting from oxidation. These anti-nitrotyrosine antibodies are not detection means specific to osteoarticular pathologies and only make it possible to demonstrate, or even treat, without distinction, cancer, Alzheimer's disease, Parkinson's disease, intestinal inflammatory diseases, lupus erythematosus, arthrosis or rheumatoid polyarthritis. These anti-nitrotyrosine antibodies indiscriminately target all the proteins comprising the amino acids of nitrotyrosine type, such that it is not possible to identify the tissue or the protein detected.

In the same way, the application PCT WO-A-98/29452 discloses an antibody specifically recognizing a nitrosylated protein, in particular nitrosylated albumin.

In these two PCT applications, the biological or biochemical marker is nitrotyrosine independently of the adjacent amino acid sequence.

The patent application PCT WO-A-03/076946 discloses a biological or biochemical marker constituted, for example, by a peptide sequence of the type HRGYPGLDG [SEQ ID No. 13] in which the tyrosine Y residues are nitrosylated or nitrated, or by a sequence LQYMRA [SEQ ID No. 14]. These two peptide sequences bearing nitrotyrosine are specific to type II collagen, which is the main component of the extracellular matrix of the cartilage synthesized by the chondrocytes.

However, the degradation of the cartilaginous tissue is not the earliest sign of the joint pathologies.

A significant need nevertheless exists, with regard to diagnosis, monitoring, prognosis and therapeutic uses, for an early or pre-symptomatic marker of joint pathology, since it is difficult, as stressed above, to rapidly diagnose such diseases, the pain, stiffness of the joint and radiography used at present being late signs of such joint diseases.

And at present, to the applicant's knowledge, the only marker specific to synovial tissue which is an early pre-symptomatic indicator of osteoarticular pathology, is glycosylated pyridinoline and more particularly diglycosylated pyridinoline (Pyr-Gal-Glc), known as glusosyl-galactosyl-pyridinoline or disaccharide pyridinoline. This early marker which is specific to synovial pathology as well as the methods and kits utilizing said marker are described in the French patent application FR-A-2 795 823. The disaccharide pyridinoline (Pyr-Gal-Glc) is specific to synovial collagen, i.e. a type I, III, IV, V or VI collagen or of a mixture thereof, preferably mainly type I and III collagens.

The documents US-A-2003/119058, U.S. Pat. No. 6,355,442, U.S. Pat. No. 6,342,361, U.S. Pat. No. 6,323,314, U.S. Pat. No. 6,110,689 disclose methods for the assay of type III collagen degradation products, for the purposes of diagnosing disorders linked to the metabolism of the collagen and, more particularly, to the bone disease known as osteoporosis. The collagens concerned are in particular type I, II and III collagens. The peptide sequences used as markers and disclosed in these documents include the peptide sequence located at the N-terminal end of a telopeptide of a type III collagen: DVKSGV [SEQ ID No. 12]. The bone pathology to which these patents relate, namely osteoporosis, clearly differs from the assorted inflammatory joint pathologies of oxidative degradations. The SEQ ID No. 12 contains no nitrosylated tyrosine.

Similarly, EP-B-0 362 235 describes a method for the analysis of type III collagen decomposition products, a process involving an antibody specific to the amino-terminal telopeptide of type III collagen. This antibody is capable of recognizing and binding to the peptide sequence constituting the whole amino-terminal telopeptide. The detection of the amino-terminal telopeptide of type III collagen according to EP-B-0 362 235 is not linked to the demonstration of oxidative inflammatory osteoarticular pathologies.

### PROBLEM TO BE SOLVED—OBJECTIVES

Generally, one of the major objectives of the invention is to provide tools and methods making it possible to improve the diagnosis, treatment, monitoring and prognosis of diseases which are accompanied by an oxidative degradation of tissues containing type III collagen (said diseases being hereafter qualified as O.D.T.Coll.III), in particular inflammatory diseases such as the osteoarticular diseases.

More specifically and given the medical benefit provided by a specific and early or pre-symptomatic marker of osteoarticular pathologies, one of the essential objectives of the present invention is to provide a novel marker of this type capable of being an alternative to the disaccharide pyridinoline (Pyr-Gal-Glc) disclosed for this purpose in the patent application FR-A-2 795 823.

It will in fact be recalled that in certain osteoarticular pathologies (in particular rheumatoid polyarthritis), the non-mineralized tissues (synovial, cartilage) are degraded before the bone matrix.

In order to further improve the specificity of the specific and early or pre-symptomatic markers of osteoarticular pathologies (in particular synovial), it would be useful to have a still more specific marker of this type of inflammatory joint disease. This is another essential objective of the present invention.

Another essential objective of the invention is to provide a specific and early or pre-symptomatic marker of the oxidative metabolism of the synovial tissue and more particularly of the nitrolysation of the synovial collagen.

Another essential objective of the invention is to provide a specific and early or pre-symptomatic marker of the oxidative metabolism of tissues containing type III collagen, such as the synovial tissue and more particularly of the nitrosylation of a type III collagen.

Another essential objective of the present invention is to provide a novel peptide marker, on the one hand simple, sensitive and reliable and, on the other hand, specific to the O.D.T.Coll.III pathologies, including the osteoarticular pathologies, more precisely the oxidative metabolism of the tissues containing collagen, such as the synovial tissue and, still more precisely, the nitrolysation of the collagen III e.g. synovial, in order to improve the earliness of the clinical information capable of being obtained for O.D.T.Coll.III pathologies, in particular osteoarticular, in order ultimately to allow better diagnosis, better monitoring, better prognosis and better treatment of said pathologies.

Another objective of the invention is to improve the methods for diagnosis of the O.D.T.Coll.III diseases, in particular osteoarticular, in particular synovial diseases.

Another essential objective of the invention is to provide novel methods for diagnosis which are economical, efficient, reliable and which can be simply carried out in vitro starting from a biological sample capable of containing degraded protein residues, in particular topical collagen residues, from the O.D.T.Coll.III diseases, in particular osteoarticular.

Another essential objective of the invention is to provide the means which are useful for these diagnosis methods, in particular the specific markers (peptide sequences, preferably collagen) and the elements for detection (antibodies) of said markers.

Another essential objective of the invention is to provide improved methods for the monitoring and prognosis of O.D.T.Coll.III pathologies, in particular osteoarticular [osteoarticular, in particular synovial] which are early, reliable, efficient, economical, and which be can be carried out simply in vitro, starting from a biological sample from an individual which might contain protein residues, in particular collagen, from degradation accounting for the pathological state.

Another essential objective of the invention is to provide improved methods for determining the effectiveness or toxicity of a medicament suitable for the treatment of an O.D.T.Coll.III pathology, in particular osteoarticular (e.g. synovial).

Another essential objective of the present invention is to provide kits for the implementation of the methods referred to in the objectives above.

Another essential objective of the invention is to provide elements for the detection, in particular of the antibodies specific to the abovementioned marker and capable of being contained in the means of diagnosis, monitoring, prognosis or determination of the toxicity or effectiveness of a remedy against these O.D.T.Coll.III pathologies, in particular synovial or osteoarticular.

Another essential objective of the invention is to provide peptide sequences specific to the diseases involving post-translational modifications, degradation of proteins, for example collagen, in particular type III collagen under the effect of a hyperoxidizing species, in particular nitrogen monoxide NO and its derivatives (O.D.T.Coll.III diseases such as the arthritis, cancer, neurodegenerative diseases).

Another essential objective of the invention is to propose the use of these peptide sequences for rapid and early in vitro determination of the existence of pathologies resulting in an oxidative stress of the synovial tissue.

Another essential objective of the invention is to provide nucleic sequences for the abovementioned peptide markers of oxidative degradation.

### BRIEF DESCRIPTION OF THE INVENTION

In order to achieve these objectives, among others, it is to the inventors' credit to have detected a novel peptide marker (IIINys) located at the N-terminal telopeptide of type III collagen. This novel marker is specific to the oxidative metabolism of tissues containing type III collagen such as the synovial tissue and more particularly to the nitrolysation of collagen III, in particular synovial.

Knowing that tissues containing type III collagen such as synovial tissue are the first to be degraded in the O.D.T.Coll.III pathologies in particular the osteoarticular diseases, this novel marker is an early marker or also a pre-symptomatic marker of said pathologies.

This novel marker is all the more useful as it can be detected qualitatively and quantitatively in vitro in biological samples (blood, urine), obtained from individuals suffering from pathologies which are accompanied by nitrolysation-type oxidative degradation of the amino-terminal telopeptides of type III collagen, such as the osteoarticular diseases, in particular synovial. This detection in vitro can be carried out simply, rapidly and economically, using antibody-type detection elements, utilizing means for revealing the antibody/antigen complex, bearing at least one epitope constituted by the marker.

The novel marker according to the invention can therefore be advantageously combined with detection kits, with antibody-type detection means and with means of production of said antibodies (cells-hybridomas-nucleic sequences).

It therefore follows that in the first of its aspects, the present invention relates to a novel marker specific to an oxidative degradation of tissues containing type III collagen, preferably to nitrosylation of the type III collagen, characterized in that it comprises at least one peptide sequence SP* of 5 to 25 amino acids (AAs), preferably of 5 to 20 AAs, and, still more preferentially, of 4 to 15 AAs, including the sub-sequence QYDSYD [SEQ ID No. 1] in which at least one of the Ys is nitrosylated* and where Q corresponds not only to glutamine in accordance with the international code but also to pyroglutamic acid.

The presence, or even the concentration of such a marker in a biological sample from an individual, provides early and reliable information on oxidative damage in a tissue from this individual, damage most often associated with inflammation. In other words, this marker peptide sequence SP* in nitrosylated form, constitutes a pointer to the pathological state of an individual suffering from or capable of suffering from O.D.T.Coll.III diseases, in particular inflammatory, such as the osteoarticular diseases, cancer, neurodegenerative diseases (Parkinson's, Alzheimer's).

Preferably, in the case where the tissues containing type III collagen and having undergone or undergoing oxidative degradation are synovial tissues, the collagen targeted by the marker is associated with synovial pathologies, i.e. pathologies corresponding to an increase in turnover, proliferation, degradation, inflammation, destruction, decomposition, pathological remodelling or degradation of the synovial membrane or of the synovial collagen.

Synovial collagen signifies, for example, within the meaning of the present disclosure, a type I, III, IV, V or VI collagen, or a mixture of these collagens. Preferably, it is a type III collagen.

The marker according to the invention is a marker specific to synovial pathology making it possible to distinguish the latter from other pathologies and in particular the pathologies affecting bone and/or cartilage.

Apart from the synovial pathologies, the invention also relates to other osteoarticular pathologies, including those which lead to a proliferation of the synovial membrane and impairment of the cartilage. These osteoarticular pathologies can be inflammatory rheumatism, metabolic arthropathies, the degenerative rheumatism, rheumatoid polyarthritis, spondylarthropathies, gout, chondrocalcinosis or arthrosis, among others.

### DETAILED DESCRIPTION OF THE INVENTION

According to a preferred characteristic of the invention, the peptide sequence SP* of the marker is chosen from the following group of sequences:

| | |
|---|---|
| QY*DSY*DVKSG; | [SEQ ID No. 2] |
| QY*DSYDVKSG; | [SEQ ID No. 3] |
| QYDSY*DVKSG; | [SEQ ID No. 4] |

the homologous sequences having a degree of homology with SEQ ID No. 2 to 4 greater than or equal to 95%, preferably 98%, and still more preferentially 99%; sequences in which Y* corresponds to a nitrosylated Y.

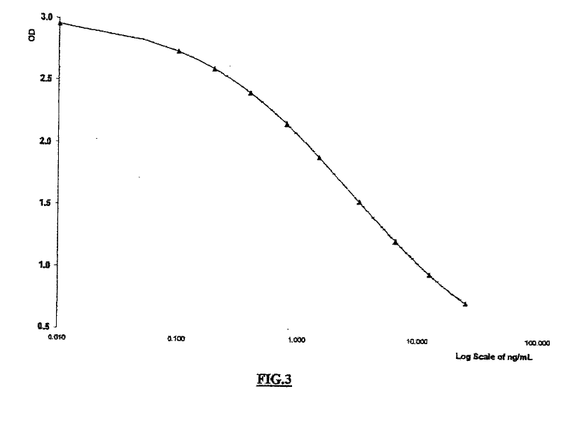

Within the meaning of the invention, the term “nitrosylated” corresponds, for example, to the post-translational modification that tyrosine undergoes in order to become 3-nitrotyrosine. This example of nitrosylation is illustrated by the attached FIG. 2.

According to a variant, the peptide sequence SP* is chosen from the following group of sequences:

| | |
|---|---|
| QY*DSY*DVKS; | [SEQ ID No. 5] |
| QY*DSYDVKS; | [SEQ ID No. 6] |
| QYDSY*DVKS; | [SEQ ID No. 7] |

the homologous sequences having a degree of homology with SEQ ID No. 5 to 7 greater than or equal to 95%, preferably 98%, and still more preferentially 99%; sequences in which Y* corresponds to a nitrosylated Y.

The marker according to the invention can comprise several peptide sequences SP* of different kinds, for example a mixture constituted by at least two of the sequences SEQ ID No. 2 to SEQ ID No. 7.

It is also possible to envisage, according to the invention, that the marker comprises in addition to the peptide sequence or sequences SP*, a non-nitrosylated peptide sequence SP, preferably the following peptide sequence: QYDSYDVKSG [SEQ ID No. 8].

It is to be noted that the peptide sequences SP*, in particular SEQ ID, No. 2, 3, 4, 5, 6 and 7 comprise a lysine residue capable of forming a bridge with other lysine residues belonging to a peptide chain of the same protein molecule or to another protein molecule.

Moreover, the bridge can involve two proteins, for example collagen proteins, which are identical or different.

In the case where these two proteins are identical, the peptide sequence SP* or SP (contained in a protein P) is linked by at least one bridge to at least one other peptide sequence SP* or SP (contained in the same molecule of protein P) or to another peptide sequence SP′ (contained in another molecule of protein P), said bridge preferably linking a lysine residue of one sequence to a lysine residue of the other sequence, and, still more preferentially, said bridge comprising a direct covalent bond or at least one pyrodinoline residue.

In the case where these two proteins are different, the peptide sequence SP* or SP (contained in a protein P) is linked by at least one bridge to at least one other peptide sequence SP^{x} (contained in a protein P^{x} different from P), said bridge preferably linking a lysine residue of one sequence to a lysine residue of the other sequence, and, still more preferentially, said bridge comprising a direct covalent bond or at least one pyrodinoline residue.

In a preferred embodiment of the invention, the peptide sequence SP*, or even the peptide sequence SP is (are) contained in a protein P constituted by type III collagen, preferably in an (advantageously aminoterminal) telopeptide of said type III collagen. On the other hand, as indicated above, the protein P^{x}, optionally linked by a bridge to the protein P carrying the sequence SP* and optionally SP, is preferably a collagen of a type different from type III, and, still more preferentially, a type I collagen, SP^{x} being preferably contained in an (advantageously aminoterminal) telopeptide.

The peptide sequence SP′, which is different from SP* and from SP, can be located anywhere in the protein P, i.e. in the triple helix type or in the telopeptides when it is collagen. The location of SP can, for example, be in the C-terminal part of the triple helix of type III collagen.

By “protein”, is meant within the meaning of the invention, the whole protein, for example the collagen or fragments of the latter resulting from oxidative degradation linked to an O.D.T.Coll.III pathology, for example an inflammatory joint pathology.

According to a second of its aspects, the present invention relates to a peptide sequence isolated from the human body or otherwise produced by a technical process, characterized in that it comprises 5 to 25 amino acids (AAs), preferably 5 to 20 AAs, and, still more preferentially, 4 to 15 AAs, including the sub-sequence QYDSYD [SEQ ID No. 1] in which at least one of the Ys is nitrosylated* and where the Q can be pyroglutamic acid.

Preferably, this peptide sequence is characterized in that it is chosen from the following group of sequences:
SP* as defined above,
SP as defined above,
SP^{x} as defined above,
   the homologous sequences having a degree of homology with the SEQ SP, SP*, SP^{x} greater than or equal to 95%, preferably 98%, and, still more preferentially 99%; in which Y* correspond to a nitrosylated Y.

This second aspect of the invention relates to the peptide sequences as such, as novel products, and useable in particular as markers specific to an oxidative degradation of tissues containing collagen III, for example the synovial tissue.

According to a third aspect, the invention relates to the genetic tools for synthesis of the peptide sequences referred to above, and in particular the sequences SP* or SP, still more particularly the sequences No. 2, 3, 4, 5, 6 and 7. In this third aspect, the invention therefore relates to any nucleic sequence coding for the marker or sequence defined above.

In the fourth of its aspects, the present invention relates to the use of at least one peptide sequence SP* or SP as defined above in the presentation of the marker according to the first aspect of the invention, or of a peptide sequence as such according to the third aspect of the invention (for example SEQ ID No. 1, 2, 3, 4, 5, 6 and 7), for a technical application chosen from the group comprising:
the diagnosis of a pathology which is accompanied by an oxidative degradation of tissues containing type III collagen;
the monitoring of a pathology which is accompanied by an oxidative degradation of tissues containing type III collagen;
the determination of the effectiveness of a medicament suitable for the treatment of a pathology which is accompanied by an oxidative degradation of tissues containing type III collagen;
or the determination of the toxicity of a medicament suitable for the treatment of a pathology which is accompanied by an oxidative degradation of tissues containing type III collagen.

Preferably, the pathology which is accompanied by an oxidative degradation of tissues containing type III collagen (O.D.T.Coll.III pathology), is a synovial pathology or another osteoarticular pathology.

The use according to the fourth aspect of the invention is to be linked directly to the fifth aspect of the present invention, namely:

→on the one hand, a method for the diagnosis, monitoring or prognosis of a pathology which is accompanied by an oxidative degradation of tissues containing type III collagen, characterized in that it comprises the following essential stages:
a. quantitative and/or qualitative in vitro analysis of a biological sample from an individual, in order to detect a marker specific to an oxidative degradation of the synovial tissue according to any one of claims 1 to 8;
b. comparison of the concentration of marker optionally detected in the sample with a reference concentration corresponding to the existence (at a determined stage) or to the absence in an individual, of a synovial pathology or another osteoarticular pathology;
   →and, on the other hand, a method for determining the effectiveness or toxicity of a medicament suitable for the treatment of a pathology which is accompanied by an oxidative degradation of tissues containing type III collagen, characterized in that it comprises the following essential stages:
a. quantitative and/or qualitative in vitro analysis of a biological sample from an individual, in order to detect a marker specific to an oxidative degradation of the synovial tissue according to any one of claims 1 to 8;
b. comparison of the concentration of marker optionally detected in the sample with a reference concentration corresponding to the existence (at a determined stage) or to the absence in an individual, of a synovial pathology or another osteoarticular pathology.

Preferably, the pathology which is accompanied by an oxidative degradation of tissues containing type III collagen (O.D.T.Coll.III pathology), is a synovial pathology or another osteoarticular pathology.

The methods according to the invention are medical methods for the prevention and treatment of pathologies which are accompanied by an oxidative degradation of tissues containing type III collagen (O.D.T.Coll.III pathology), preferably synovial pathologies or osteoarticular pathologies.

They involve detecting in biological samples, the specific marker according to the invention as defined above. A biological sample can comprise in particular a biological fluid, in particular those which are usually tested as clinical samples. This includes all body fluids such as blood (in all analyzable forms, in particular serum and plasma), urine, saliva, sweat, culture or cell extracts or supernatants or synovial fluid.

Synovial fluid is advantageous for the detection of synovial pathologies to the extent that it is specific to the synovial tissue or synovial membrane. A biological sample can be also a tissue extracted from the individual, this tissue being optionally cultured before the detection of the marker is carried out on this tissue. This tissue can be for example the synovial membrane.

In the methods according to the invention, the detection of the marker in the biological sample is qualitative and/or quantitative. This detection can be carried out for example by a high performance liquid chromatography (HPLC) technique, by fluorescence, by ultraviolet spectroscopy, by electrochemical detection or by proteomic analysis or “microarray” techniques.

Preferably, the detection is rather based on an immunological technique (e.g. ELISA, immuno-enzymatic technique, immuno-fluorescence technique, radio-immunological technique, chemo-immunological technique).

These different detection techniques are well known to a person skilled in the art. With regard to the immunological techniques, reference can be made to Diamantis and Cristopoulos, Immunoassay, Academic Press, San Diego 1996, in particular pages 579 et seq. For the proteomic analysis technique, reference may in particular be made to Urbanowska et al. Development of protein microarray technology to monitor biomarkers of rheumatoid arthritis disease. Cell Biol. Toxicol, 2003 19(3): 189-202 to Glokler and Angenendt. Protein and antibody microarray technology. Journal of Chromatography B, 2003 797(1-2): 229-240.

Concerning the “reference concentration” which serves as a basis for comparison in the methods according to the invention, the following stipulations can be applied.

The choice of the reference concentration corresponding either to an absence of pathology, or to the existence of a pathology at a determined stage, can be made by a person skilled in the art depending on the selected marker as well as the technique used.

By way of example, the reference concentration which delimits the pathological state from the non-pathological state for different techniques, corresponds to the average of patients above the 95^{th} percentile of the control values.

Since the invention in its fifth aspect relates to methods involving the detection of the marker, it is clear that a sixth aspect of the invention relates to the means allowing the implementation of these methods.

Thus, the invention relates to a means for the diagnosis, monitoring or prognosis of a pathology which is accompanied by an oxidative degradation of tissues containing type III collagen, characterized in that it comprises at least one element for detection of the marker as defined above.

Another means according to the invention is a means for determining the effectiveness of a medicament suitable for the treatment of a pathology which is accompanied by an oxidative degradation of tissues containing type III collagen, characterized in that it comprises at least one element for detecting the marker as defined above.

Another means according to the invention is a means for determining the toxicity of a medicament suitable for the treatment of a pathology which is accompanied by an oxidative degradation of tissues containing type III collagen, characterized in that it comprises at least one element for detection of the marker as defined above.

As indicated above within the framework of the recapitulation of the methods, the preferred detection according to the invention is an immunological type detection.

Thus, according to the invention, the abovementioned means have the characteristic that the detection element as defined above is characterized in that the detection element comprises at least one (purified) antibody capable of recognizing the epitope included in the peptide sequence SP* of the marker as defined above and of specifically binding to the protein carrying said marker or said sequence.

The means of the invention apply to pathologies which are accompanied by an oxidative degradation of tissues containing type III collagen (O.D.T.Coll.III pathology) and, preferably, to synovial pathologies or other osteoarticular pathologies.

In a seventh aspect, the invention relates, as a novel product per se, to a (purified) antibody capable of recognizing the epitope included in the peptide sequence SP* of the marker as defined above or the sequence as defined above and capable of specifically binding to the protein carrying said marker or said sequence.

By “antibodies” within the meaning of the present invention, is meant, for example, the monoclonal and polyclonal antibodies or any other fragment derived from the antibodies, humanized antibodies, Fab fragments, chimeric antibodies or any other fragment of a specific antigen-antibody complex, among other things. When the antibody is used within the context of the method for detection of a fragment of collagen (protein P), the expression “antibody” comprises any analyte produced which is specific to the fragment of collagen containing at least the SEQ ID No. 1.

The term “epitote” denotes for example a minimum sequence of amino acids recognized by the antibody in the carrier protein P and/or in the peptide sequences SP*, or even SP. The epitote determines the immunospecificity. Within the context of the invention, a epitote can be a short sequence of three contiguous amino acids or a longer sequence containing at least four amino acids.

Once the protein P within the meaning of the present invention can contain protein fragments, the latter can be polypeptides, i.e. peptide molecules containing more than 50 amino acids.

According to a preferred characteristic of the invention, the antibodies useful as detection elements in the methods or means considered, can be monoclonal or polyclonal. The production of these antibodies is carried out according to the standard methods well known to a person skilled in the art and described in particular by HARLOW and LANE in 1988, as well as by KÖHLER and MILSTEIN in 1975 in “Continuous cultures of fused cells secreting antibody of predefined specificity” Nature 1975, 256(5517):495-7. In particular, the markers to be detected will for example be prepared, according to standard peptide syntheses, which will be coupled to a so-called “carrier” protein which is preferentially KLH (Keyhole limpet hemocyanine) or bovine or human serum albumin, ovalbumin, tyroglobulin or any other “carrier” protein such as edestin, tetanus or cholera toxin, polyamino acids such as poly-D-Lysine-D glutamic acid. This coupling is carried out via a cysteine, glutaraldehyde, or carbodiimide for example. This coupled sequence then allows the immunization of mice, rats, rabbits, chickens, or any other individual allowing the production of antibodies.

The antibodies according to the invention are specific as they recognize the degradation fragments of the type III collagen containing at least four consecutive amino acids of SEQ ID No. 1, preferably at least five, present in the aminoterminal telopeptide of type III collagen.

The obtaining of an antibody specific to the peptide markers SP*, or even SP according to the invention, can require the antigen to be, in a first stage, extracted and purified from urine, for example. Such an extraction or purification can be advantageously carried out by the technique described in the example and also by the synthesis of synthetic peptides.

It should be noted that, according to an advantageous feature of the invention, the means which are useful in the methods according to the invention, in particular the detection elements that are for example the antibodies specific to the markers, have the property of detecting the bridges between at least two peptide sequences SP or between at least one peptide sequence SP* and at least one peptide sequence SP, or also at least one peptide sequence SP* and at least one other protein P^{x}. These bridges are produced preferably between lysyl residues of the different peptide sequences in question.

At the least, the protein P is preferably the type III collagen and the protein P^{x} is preferably a type I collagen.

In its eighth aspect, the invention relates, on the one hand, to a kit for the diagnosis, monitoring or prognosis of a pathology which is accompanied by an oxidative degradation of tissues containing type III collagen, and on the other hand, a kit for determining the effectiveness or toxicity of a medicament suitable for the treatment of a pathology which is accompanied by an oxidative degradation of tissues containing type III collagen.

These two types of kit have the characteristic of comprising at least one means as defined above.

These kits comprise detection or reactive elements (for example antimarker antibodies as defined above) intended to be brought into contact with the biological sample to be analyzed. These kits also comprise means for revealing the formation of the antigen/antibody complexes (for example secondary antibodies, coloured indicators, fluorescent indicators, radioactive indicators, etc.). In a standard fashion, an operating protocol is also provided in these kits, making it possible to carry out the analysis, the reading of the results revealed by the revealing means is carried out with the naked eye for a qualitative analysis and using more sophisticated measuring instruments for the quantitative assays.

Preferably, these kits relate to the synovial pathologies or other osteoarticular pathologies.

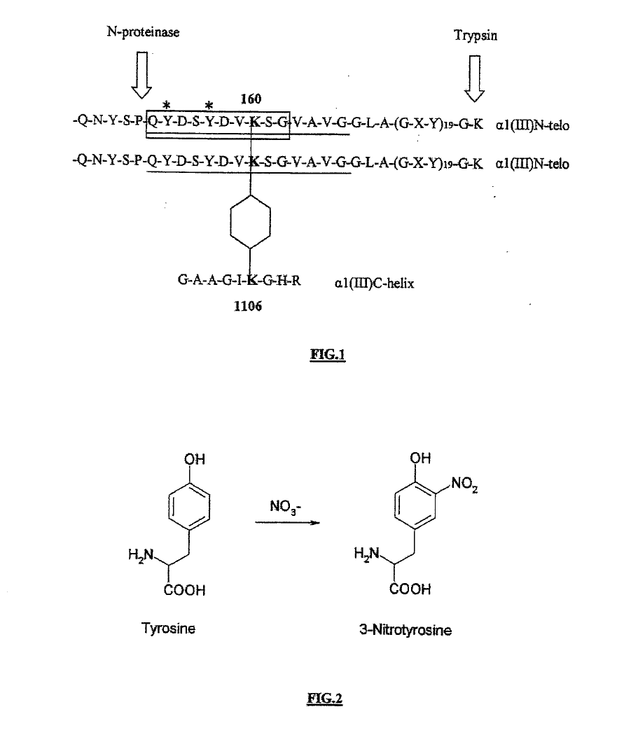

The invention will be better understood and its advantages will become apparent from the following examples, which describe the preparation of an example of a marker according to the invention, the preparation of detection elements constituted by polyclonal antibodies specific to said marker and a method for detection by an immunological technique of competitive ELISA type of the marker according to the invention, and finally the demonstration of the detection of patients suffering from rheumatoid polyarthritis on serum samples originating from these patients by means of the marker and the method according to the invention. The attached FIGS. 1, 2, 3, 4, 5, 6A and 6B complete the information given by the following examples.

### DESCRIPTION OF FIGS. 1, 2, 3, 4, 5, 6A and 6B

FIG. 1 represents the structure of the aminoterminal telopeptide of the type III collagen bridged with two other molecules of type III collagen by pyridinoline or one of its derivatives. The boxed sequence is SEQ ID No. 2 which corresponds to a preferred embodiment of the marker according to the invention, hereafter called IIINys. The asterisks indicate the two tyrosine residues which are nitrosylated.

FIG. 2 represents the formation of 3-nitrotyrosine by the action of peroxynitrite on tyrosine.

FIG. 3 represents the standard ELISA-type curve assaying the IIINys peptide of sequence SEQ ID No. 2

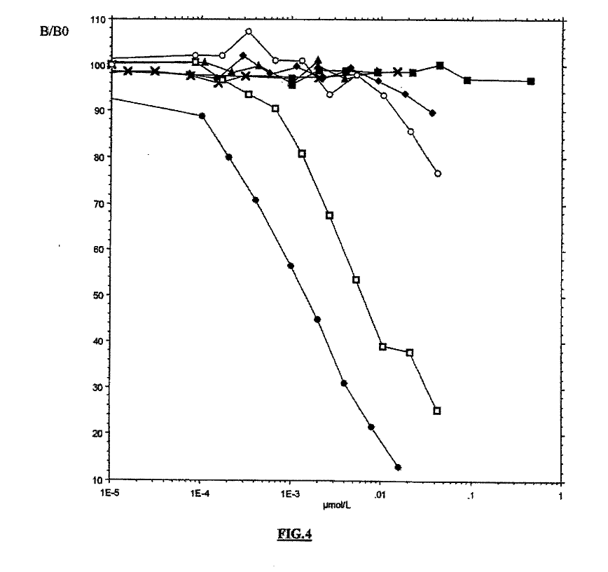

FIG. 4 illustrates the specificity of the polyclonal antibodies prepared vis-à-vis the IIINys, with respect to other synthetic peptides. B/B0 represents the ratio between the quantity of anti-IIINys antibodies fixed to the antigen fixed (“coated”) onto the plate in the presence of competitor compound (B) and in the absence of competitor compound (B0).

| | |
|---|---|
| -QY*DSY*DVKSG | SEQ ID No. 2 |
| ▴-QYDSYDVKSG | SEQ ID No. 8 |
| ▪-3-nitro tyrosine | SEQ ID No. 12 |
| X-nitrated BSA | SEQ ID No. 11 |
| ♦-PQY*DSY*DVKSG | SEQ ID No. 9 |
| □-QY*DSYDVKSG | SEQ ID No. 3 |
| ◯-QYDSY*DVKSG | SEQ ID No. 4 |

FIG. 5 shows the serum concentration C of IIINys (μg/L) in healthy adults (n=29) [CTRL] and patients suffering from rheumatoid polyarthritis (n=30) [RA].

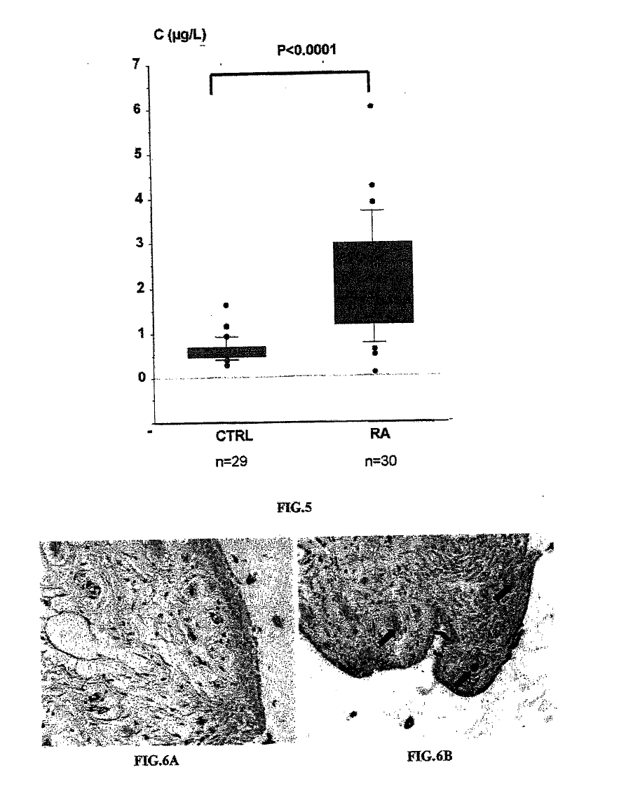

FIG. 6A represents an immunohistochemical analysis of sections of synovial tissues from patients suffering from arthrosis of the knee marked with the pre-immune serum from a rabbit having produced the IIINys antiserum (serum taken before immunization and serving as a negative control).

FIG. 6B shows an immunohistochemical analysis of sections of synovial tissues from patients suffering from arthrosis of the knee marked with the IIINys antibody. The arrows noted on the figure show the marking by the IIINys antibody.

### EXAMPLES

By way of a non-limitative illustration of the invention, FIG. 1 shows the peptide sequences of two aminoterminal telopeptides of a type III collagen, one of which comprises a peptide sequence SP* of SEQ ID No. 2 constituting a preferred embodiment of the marker IIINys according to the invention, these two telopeptides being moreover linked by a bridge with the triple helix part of another molecule of type III collagen.

The non-helical telopeptide region is underlined. The arrows represent the trypsin and N-proteinase cleavage sites. The asterisks above the Y codes indicate the tyrosine residues or the nitrosylated tyrosine residues. The bridge is a pyridinoline-type bridge linking the two telopeptides of a molecule of type III collagen with the C-terminal region of the helix of another molecule of type III collagen.

### A—Methods

### 1. Preparation of the Synthetic Peptide

The preparation of the synthetic peptides can be carried out according to the techniques widely described in the scientific literature. The peptide IIINys of sequence SEQ ID No. 2 derived from the sequence of the N-terminal telopeptide of type III collagen (GenBank accession number: P02461: SEQ ID No. 10) was produced by the solid-phase peptide synthesis technique using reversible blocking of the amine of the amino acid by Fmoc (9-Fluorenylmethyloxycarbonyl). The purity of the peptide was verified by HPLC and by mass spectrometry.

The peptide serving as immunogen, synthesized with an additional cysteine on the C-terminal side, was coupled on the C-terminal side to a carrier protein which is KLH (keyhole limpet hemocyanine) which will allow a better immunological response vis-à-vis this peptide. The synthetic peptide which is adsorbed on the microplates was coupled to biotin (Harlow and Lane, 1988).

### 2. Preparation of the Polyclonal Antibodies

Rabbits were injected by intra-peritoneal route with the peptide conjugated to KLH emulsified with Freund's complete adjuvant. This operation was repeated 3 times over three months using the same peptide with Freund's incomplete adjuvant. After each injection a sample of rabbit blood was taken. This blood was left to coagulate for 30 minutes and the whole sample was centrifuged for 10 minutes at 2500 rpm in order to recover the serum. Ten days after the last injection, the rabbits were sacrificed and all the blood was recovered. Each serum sample was titrated by ELISA technique in the presence of the synthetic peptide. The antiserum with the best titre was selected for the finalization of the competitive ELISA (Harlow and Lane, 1988a).

### 3. Titration of the Antisera

The titres of the antisera were produced by adsorbing the peptide conjugated to the biotin diluted with the coating buffer on streptavidin plates (Nunc, Denmark) for 2 hours at ambient temperature. After washing, 50 μl of antisera diluted from 1/100 to 1/1,000,000 are added to 50 μl of dilution buffer in the wells and incubated overnight at 4° C. After washing, 100 μl of secondary antibodies coupled to the peroxidase directed against the rabbit immunoglobulins are added over one hour at ambient temperature. The wells are washed and 100 μl of substrate (TMB) are added. The reaction is stopped with 100 μl of H₂SO₄.

### 4. Competitive ELISA for the Peptide IIINys of the Aminoterminal Telopeptide Alpha 1 Chain of the Type III Collagen

The peptide conjugated to the biotin diluted with the coating buffer is adsorbed on plates avidinated for 2 hours at ambient temperature. After washing, 50 μl of antisera diluted to the optimum titre are added to 50 μl of standard (synthetic peptide diluted in buffer) or to 50 μl of samples to be assayed and are left to incubate overnight at 4° C. After washing, 100 μl of secondary antibodies coupled to the peroxidase directed against the rabbit immunoglobulins are added over one hour at ambient temperature. The wells are washed and 100 μl of substrate (TMB) are added. The reaction is stopped with 100 μl of H₂SO₄.

5. Immunohistochemical Analysis of Synovial Tissue Sections from Patients Suffering from Osteoarthritis

Specimens of the synovial membrane of patients suffering from arthrosis were taken during knee replacement surgery. The synovial membrane specimens were fixed and embedded in paraffin according to usual laboratory techniques. Immunohistochemistry was carried out on 7 82 m sections with diaminobenzidine (standard Envision™+HRP protocol). Briefly, the sections are dewaxed, the endogenous peroxidases are blocked and the antigen is exposed by 2 hours' incubation at 60° C. in tris-EDTA buffer (pH 9). The non-specific sites are blocked with caseine in TBS. The sections are subsequently incubated overnight at 4° C. with the antibodies directed against the sequence SEQ ID No. 3 and non-immune antibodies as negative control (originating from the same rabbit before immunization). After careful washings, an Envision™ anti-rabbit antibody coupled to HRP (DAKO) is added to the sections and left to incubate for 30 minutes at ambient temperature.

The excess Envision™ antibody is eliminated by washings and the solution of substrate comprising diaminobenzidine (DAB+, DAKO) is added for the reaction with peroxidase producing a brown colour. The marking with DAB is activated by copper sulphate (CuSO₄) and the double marking was carried out with Mayers hematoxylin acid which stains the cell nuclei.

### B—Results

### Example 1

### Competitive ELISA for the Peptide IIINys of the N-telopeptide of Type III Collagen

The methods for production of the peptides and antisera as well as the competitive ELISA method are described above.

A standard or calibration curve was obtained on a semi-log graph by assaying the 8 standards (from 0.2 ng/ml to 25 ng/ml) with the antiserum directed against the sequence SEQ ID No. 2 selected for its titre (FIG. 3). The peptide concentration of sequence SEQ ID No. 2 in the samples is obtained by extrapolation of the calibration curve (Optical Density OD of the 8 standards assayed as a function of the Log of their concentrations in ng/mL: “Log scale of ng/mL”).

The detection limit of this test was determined by calculating the average of 32 determinations of the standard 0 from which three times the standard deviation is subtracted. The detection limit is 0.16 ng/mL.

### Example 2

### Characterization of the Specificity of the Antiserum anti IIINys

The antiserum selected was tested vis-à-vis different peptides according to the ELISA method described previously in order to verify its specificity (FIG. 4). The peptides tested are: the peptide IIINys of sequence SEQ ID No. 2 (the immunogenic peptide), the peptide of sequence SEQ ID No. 3 (the peptide IIINys with only the first tyrosine nitrosylated), the peptide of sequence SEQ ID No. 4 (the peptide IIINys with only the second tyrosine nitrosylated), the peptide of sequence SEQ ID No. 9 (the peptide IIINys with an additional amino acid on the N-terminal side), the peptide of sequence SEQ ID No. 8 (the peptide IIINys with its tyrosines non nytrosylated), 3-nitrotyrosine (FIG. 2), with the nitrosylated BSA of sequence SEQ ID No. 11. The peptides of sequence SEQ ID No. 8 and 9 as well as the nitrosylated BSA and 3-nitrotyrosine are not capable of entering into competition with the peptide IIINys (SEQ ID No. 2) adsorbed on the plate. By contrast, the antiserum recognizes the peptides of sequence SEQ ID No. 3 and 4 but with less affinity than for the peptide IIINys. Therefore, the antiserum anti-IIINys is specific to the N-proteinase cleavage site on the N-terminal side of the N-telopeptide of the type III collagen and is also more specific to the first nitrosylation.

### Example 3

### The Level of Serum IIINys is Increased in Patients Suffering from Rheumatoid Polyarthritis Compared with Healthy Patients

Sera from healthy patients (n=29) and from patients suffering from rheumatoid polyarthritis (n=30) were collected and assayed for IIINys as described in the protocol. The concentration of IIINys (ng/ml) for the different populations is presented in FIG. 5. The horizontal lines represent the median of IIINys (ng/ml) in each group. The significant values for the difference between the groups (P) are shown by the graph.

### Example 4

### Immunohistochemistry on Synovial Tissues from Patients Suffering from Arthrosis

The immunohistochemistry of synovial tissues from patients suffering from arthrosis of the knee show intense marking with anti-IIINys antibodies in the extracellular matrix, more precisely around the synoviocytes and inside the macrophages (FIG. 6B) and no marking with the pre-immune serum (FIG. 6A), the arrows represent the pericellular space of the synoviocytes marked by the anti-IIINys antibody.

1-25. (canceled)

26. Marker specific to an oxidative degradation of tissues containing type III collagen, preferably to nitrosylation of type III collagen, comprising at least one peptide sequence SP* of 5 to 25 amino acids (AAs), preferably of 5 to 20 AAs, and, still more preferentially, of 4 to 15 AAs, including the sub-sequence QYDSYD [SEQ ID No. I] in which at least one of the Ys is nitrosylated* and where Q corresponds not only to glutamine in accordance with the international code but also to pyroglutamic acid.

27. Marker of claim 26, wherein the tissues referred to include synovial tissue.

28. Marker of claim 26, wherein the peptide sequence SP* is chosen:
→from the following group of sequences:

| | |
|---|---|
| QY*DSY*DVKSG; | [SEQ ID No. 2] |
| QY*DSYDVKSG; | [SEQ ID No. 3] |
| QYDSY*DVKSG; | [SEQ ID No. 4] |

the homologous sequences having a degree of homology with SEQ ID No. 2 to 4 greater than or equal to 95%, preferably 98%, and still more preferentially 99%; sequences in which Y* corresponds to a nitrosylated Y.
→and preferably, from the following group of sequences:

| | |
|---|---|
| QY*DSY*DVKS; | [SEQ ID No. 5] |
| QY*DSYDVKS; | [SEQ ID No. 6] |
| QYDSY*DVKS; | [SEQ ID No. 7] |

the homologous sequences having a degree of homology with SEQ ID No. 5 to 7 greater than or equal to 95%, preferably 98%, and still more preferentially 99%; sequences in which Y* corresponds to a nitrosylated Y.

29. Marker of claim 26, comprising the non-nitrosylated peptide sequence SP, preferably the following peptide sequence: QYDSYDVKSG [SEQ ID No. 8].

30. Marker of claim 29, wherein the peptide sequence SP* or SP (contained in a protein P) is linked by at least one bridge to at least one other peptide sequence SP* or SP (contained in the same molecule of protein P) or to another peptide sequence SP′ (contained in another molecule of protein P), said bridge preferably linking a lysine residue of one sequence to a lysine residue of the other sequence, and, still more preferentially, said bridge comprising a direct covalent bond or at least one pyrodinoline residue or one of its derivatives.

31. Marker of claim 30, wherein the peptide sequence SP* or SP (contained in a protein P) is linked by at least one bridge to at least one other peptide sequence SP^{x} (contained in a protein P^{x} which is different from P), said bridge preferably linking a lysine residue of one sequence to a lysine residue of the other sequence, and, still more preferentially, said bridge comprising a direct covalent bond or at least one pyrodinoline residue or one of its derivatives.

32. Marker of claim 30, wherein the protein P is type III collagen, SP* or SP being preferably contained in an (advantageously aminoterminal) telopeptide.

33. Marker of claim 31, wherein the protein P^{x} is collagen of a type different from type III, preferably a type I collagen.

34. Peptide sequence isolated from the human body or otherwise produced by a technical process, comprising 5 to 25 amino acids (AAs), preferably 5 to 20 AAs, and, still more preferentially, 4 to 15 AAs, including the sub-sequence QYDSYD [SEQ ID No. I] in which at least one of the Ys is nitrosylated* and where Q corresponds not only to glutamine in accordance with the international code but also to pyroglutamic acid.

35. Peptide sequence of claim 34, chosen from the following group of sequences:
SP* as defined in claim 26,
SP as defined in claim 29,
SP^{x} as defined in claim 33,
the homologous sequences having a degree of homology with the SEQ SP, SP*, SP^{x} greater than or equal to 95%, preferably 98%, and, still more preferentially 99%; in which Y* corresponds to a nitrosylated Y.

36. Nucleic sequence encoding the marker of claim 26.

37. Use of at least one peptide sequence of claim 34, for a technical application chosen from the group comprising:
the diagnosis of a pathology which is accompanied by an oxidative degradation of tissues containing type III collagen;
the monitoring of a pathology which is accompanied by an oxidative degradation of tissues containing type III collagen;
the determination of the effectiveness of a medicament suitable for the treatment of a pathology which is accompanied by an oxidative degradation of tissues containing type III collagen;
or the determination of the toxicity of a medicament suitable for the treatment of a pathology which is accompanied by an oxidative degradation of tissues containing type III collagen;

38. Use of claim 37, wherein the pathology which is accompanied by an oxidative degradation of tissues containing type Ill collagen, is a synovial pathology or another osteoarticular pathology.

39. Method for the diagnosis, monitoring or prognosis of a pathology which is accompanied by an oxidative degradation of tissues containing type Ill collagen, comprising the following essential stages:
a. quantitative and/or qualitative in vitro analysis of a biological sample from an individual, in order to detect a marker specific to an oxidative degradation of the synovial tissue of claim 26;
b. comparison of the concentration of marker optionally detected in the sample with a reference concentration corresponding to the existence (at a determined stage) or to the absence in an individual, of the pathology considered.

40. Method for determining the effectiveness or toxicity of a medicament suitable for the treatment of a pathology which is accompanied by an oxidative degradation of tissues containing type Ill collagen, characterized in that it comprises the following essential stages:
a. quantitative and/or qualitative in vitro analysis of a biological sample from an individual, in order to detect a marker specific to an oxidative degradation of the synovial tissue of claim 26;
b. comparison of the concentration of marker optionally detected in the sample with a reference concentration corresponding to the existence (at a determined stage) or to the absence in the individual, of the pathology considered.

41. Method of claim 39, wherein the pathology which is accompanied by an oxidative degradation of tissues containing type Ill collagen is a synovial pathology or another osteoarticular pathology.

42. Method of claim 40, wherein the pathology which is accompanied by an oxidative degradation of tissues containing type Ill collagen is a synovial pathology or another osteoarticular pathology.

43. Means for the diagnosis, monitoring or prognosis of a pathology, or determining the effectiveness or toxicity of a medicament suitable for the treatment of a pathology which is accompanied by an oxidative degradation of tissues containing type III collagen, comprising at least one element for detection of the marker of claim 26.

44. Means of claim 43, wherein the detection element comprises at least one (purified) antibody capable of recognizing the epitope included in the peptide sequence SP* of the marker of claim 26 and of specifically binding to the protein carrying said marker or said sequence.

45. Means of claims 43, wherein the pathology which is accompanied by an oxidative degradation of tissues containing type Ill collagen is a synovial pathology or another osteoarticular pathology.

46. Purified antibody capable of recognizing the epitope included in the peptide sequence SP* of the marker of claim 26 and of specifically binding to the protein carrying said marker.

47. Purified antibody capable of recognizing the epitope included in the peptide sequence SP* of the sequence of claim 34.

48. Kit for the diagnosis, monitoring or prognosis of a pathology or determining the effectiveness or toxicity of a medicament suitable for the treatment of a pathology, which is accompanied by an oxidative degradation of tissues containing type Ill collagen, comprising at least one means of claim 42.

49. Kit according to claim 48, wherein the pathology which is accompanied by an oxidative degradation of tissues containing type Ill collagen is a synovial pathology or another osteoarticular pathology.

50. Nucleic sequence encoding the sequence of claim 34.
